(19) United States
(12) Patent Application Publication
(10) Pub. No.: US 2009/0306151 A1
(43) Pub. Date: Dec. 10, 2009

(54) Pharmaceutical preparation containing an angiotensin II receptor antagonist and a calcium channel blocker

(75) Inventors: Takeshi Hamaura, Kanagawa (JP); Mitsuru Kanno, Kanagawa (JP)

Correspondence Address: FRISHAUF, HOLTZ, GOODMAN & CHICK, PC, 220 Fifth Avenue, 16TH Floor, NEW YORK, NY, 10001-7708, US

(73) Assignee: DAIICHI SANKYO COMPANY, LIMITED, Tokyo (JP)

(21) Appl. No.: 12/583,221

(22) Filed: Aug. 17, 2009

Related U.S. Application Data
(62) Division of application No. 11/922,543, filed on Jan. 4, 2008.

(30) Foreign Application Priority Data
Jun. 27, 2005 (JP) 2005-187251

(51) Int. Cl. A61K 31/44; A61P 9/12

(52) U.S. Cl. 514/356

(57) ABSTRACT

A pharmaceutical preparation comprising an angiotensin II receptor antagonist, a calcium channel blocker and at least one substance selected from a hydrophilic polymer, an acidic substance and a fluidizing agent. The pharmaceutical preparation demonstrates improved dissolution properties.

### CROSS-REFERENCE TO RELATED APPLICATIONS

This application is a Divisional application of application Ser. No. 11/922,543 filed Jan. 4, 2008, which is a United States national phase application of PCT/JP2006/313175. The entire contents of each of application Ser. No. 11/922,543 and PCT/JP2006/313175 are incorporated by reference herein.

### TECHNICAL FIELD

The present invention relates to a pharmaceutical preparation comprising an angiotensin II receptor antagonist and a calcium channel blocker.

### BACKGROUND ART

Currently, calcium channel blockers and angiotensin II receptor antagonists are widely used clinically as medicaments for the treatment and prophylaxis of hypertension. Since calcium channel blockers exert natriuretic action in addition to vasodilatory action, they are effective against hypertension caused by fluid retention (renin-independent). On the other hand, angiotensin II receptor antagonists are particularly effective against renin-dependent hypertension, and have excellent organ protective effects. Thus, it is expected that the combined use of a calcium channel blocker and an angiotensin II receptor antagonist should allow stable and effective antihypertensive therapy regardless of the cause of the hypertension.

A number of combination drugs comprising an angiotensin II receptor antagonist and a calcium channel blocker have been proposed in the prior art, such as Patent Documents 1 to 4 below. However, there has been no disclosure in the prior art of combination drugs comprising an angiotensin II receptor antagonist and a calcium channel blocker that additionally comprise a hydrophilic polymer, acidic substance or fluidizing agent to afford improved dissolution properties.

[Patent Document 1] International Publication WO 92/10097

[Patent Document 2] International Publication WO 92/20342

[Patent Document 3] International Publication WO 00/02543

[Patent Document 4] International Publication WO 2004/067003

### DISCLOSURE OF THE INVENTION

The object of the present invention is to provide a pharmaceutical preparation with improved dissolution properties comprising an angiotensin II receptor antagonist, a calcium channel blocker and at least one substance selected from a hydrophilic polymer, an acidic substance and a fluidizing agent.

As a result of conducting extensive research to solve the aforementioned problems, the present inventors found that a pharmaceutical preparation with improved dissolution properties is obtained by incorporation of at least one substance selected from a hydrophilic polymer, an acidic substance and a fluidizing agent in a pharmaceutical preparation comprising an angiotensin II receptor antagonist and a calcium channel blocker, thereby leading to completion of the present invention.

The present invention provides a pharmaceutical preparation comprising an angiotensin II receptor antagonist, a calcium channel blocker and at least one substance selected from a hydrophilic polymer, an acidic substance and a fluidizing agent (particularly a preparation for the prophylaxis or treatment of hypertension), the use of an angiotensin II receptor antagonist and a calcium channel blocker to manufacture the aforementioned pharmaceutical preparation (particularly a preparation for the prophylaxis or treatment of hypertension), and a method for preventing or treating a disease (particularly hypertension) in which the aforementioned pharmaceutical preparation comprising pharmacologically effective amounts of an angiotensin II receptor antagonist and a calcium channel blocker is administered to warm-blooded animals (particularly humans).

Specifically, the present invention provides:

(1) a pharmaceutical preparation comprising an angiotensin II receptor antagonist, a calcium channel blocker and at least one substance selected from a hydrophilic polymer, an acidic substance and a fluidizing agent.
(2) the pharmaceutical preparation according to (1) comprising:

(A) an angiotensin II receptor antagonist;

(B) a calcium channel blocker; and

(C) a hydrophilic polymer,

(3) the pharmaceutical preparation according to (1) comprising:

(A) an angiotensin II receptor antagonist;

(B) a calcium channel blocker; and

(C) an acidic substance,

(4) the pharmaceutical preparation according to (1) comprising:

(A) an angiotensin II receptor antagonist;

(B) a calcium channel blocker; and

(C) a fluidizing agent,

(5) the pharmaceutical preparation according to (1) to (4) wherein the angiotensin II receptor antagonist is losartan, candesartan, valsartan, telmisartan, pratosartan, olmesartan or irbesartan or a pharmacologically acceptable salt or ester thereof,
(6) the pharmaceutical preparation according to (1) to (4) wherein the angiotensin II receptor antagonist is losartan, candesartan cilexetil, valsartan, telmisartan, pratosartan, olmesartan medoxomil or irbesartan,
(7) the pharmaceutical preparation according to (1) to (4) wherein the angiotensin II receptor antagonist is olmesartan medoxomil,
(8) the pharmaceutical preparation according to (1) to (7) wherein the calcium channel blocker is nifedipine, nimodipine, nilvadipine, manidipine, barnidipine, nitrendipine, benidipine, nicardipine, lercanidipine, amlodipine, nisoldipine, efonidipine, cilnidipine, azelnidipine, felodipine, aranidipine or pranidipine or a pharmacologically acceptable salt thereof,
(9) the pharmaceutical preparation according to (1) to (7) wherein the calcium channel blocker is manidipine, barnidipine, benidipine, nicardipine, lercanidipine, amlodipine, efonidipine or azelnidipine or a pharmacologically acceptable salt thereof,
(10) the pharmaceutical preparation according to (1) to (7) wherein the calcium channel blocker is amlodipine or a pharmacologically acceptable salt thereof,
(11) the pharmaceutical preparation according to (1) to (7) wherein the calcium channel blocker is amlodipine besylate,
(12) the pharmaceutical preparation according to (1), (2) or (5) to (11) wherein the hydrophilic polymer is at least one compound selected from cellulose derivatives and synthetic polymers,
(13) the pharmaceutical preparation according to (1), (2) or (5) to (11) wherein the hydrophilic polymer is at least one compound selected from hydroxypropyl methyl cellulose, methyl cellulose, hydroxypropyl cellulose, sodium carboxymethyl cellulose, macrogol, HA Sankyo, polyvinylpyrrolidone and polyvinyl alcohol,
(14) the pharmaceutical preparation according to (1), (2) or (5) to (11) wherein the hydrophilic polymer is at least one compound selected from cellulose derivatives,
(15) the pharmaceutical preparation according to (1), (2) or (5) to (11) wherein the hydrophilic polymer is at least one compound selected from hydroxypropyl methyl cellulose, methyl cellulose, hydroxypropyl cellulose and sodium carboxymethyl cellulose,
(16) the pharmaceutical preparation according to (1), (2) or (5) to (11) wherein the hydrophilic polymer is either or both of methyl cellulose and hydroxypropyl cellulose,
(17) the pharmaceutical preparation according to (1), (2) or (5) to (11) wherein the hydrophilic polymer is macrogol,
(18) the pharmaceutical preparation according to (1), (3) or (5) to (11) wherein the acidic substance is either or both of tartaric acid and ascorbic acid,
(19) the pharmaceutical preparation according to (1), (4) or (5) to (11) wherein the fluidizing agent is at least one compound selected from calcium silicate, light anhydrous silicic acid, anhydrous calcium hydrogenphosphate, synthetic hydrotalcite and magnesium metasilicate aluminate,
(20) the pharmaceutical preparation according to (1) to (19) wherein the pharmaceutical preparation is a single dosage form,
(21) the pharmaceutical preparation according to (20) wherein the single dosage form is a solid dosage form,
(22) the pharmaceutical preparation according to (21) wherein the solid dosage form is selected from powders, grains, granules, capsules and tablets, and
(23) the pharmaceutical preparation according to (21) wherein the solid dosage form is a tablet.

In addition, a pharmaceutical preparation obtained by arbitrarily combining (1) to (23) above is also preferable, examples of which are indicated below.

(24) The pharmaceutical preparation according to (1) or (2) wherein the angiotensin II receptor antagonist is losartan, candesartan cilexetil, valsartan, telmisartan, pratosartan, olmesartan medoxomil or irbesartan,

the calcium channel blocker is amlodipine or a pharmacologically acceptable salt thereof, and

the hydrophilic polymer is at least one compound selected from hydroxypropyl methyl cellulose, methyl cellulose, hydroxypropyl cellulose, sodium carboxymethyl cellulose, macrogol, HA Sankyo, polyvinylpyrrolidone and polyvinyl alcohol,

(25) The pharmaceutical preparation according to (1) or (2) wherein the angiotensin II receptor antagonist is losartan, candesartan cilexetil, valsartan, telmisartan, pratosartan, olmesartan medoxomil or irbesartan,

the calcium channel blocker is amlodipine besylate, and

the hydrophilic polymer is at least one compound selected from hydroxypropyl methyl cellulose, methyl cellulose, hydroxypropyl cellulose, sodium carboxymethyl cellulose, macrogol, HA Sankyo, polyvinylpyrrolidone and polyvinyl alcohol,

(26) the pharmaceutical preparation according to (1) or (2) wherein the angiotensin II receptor antagonist is olmesartan medoxomil,

the calcium channel blocker is manidipine, barnidipine, benidipine, nicardipine, lercanidipine, amlodipine, efonidipine or azelnidipine or a pharmacologically acceptable salt thereof, and

the hydrophilic polymer is at least one compound selected from hydroxypropyl methyl cellulose, methyl cellulose, hydroxypropyl cellulose, sodium carboxymethyl cellulose, macrogol, HA Sankyo, polyvinylpyrrolidone and polyvinyl alcohol,

(27) the pharmaceutical preparation according to (1) or (2) wherein the angiotensin II receptor antagonist is olmesartan medoxomil,

the calcium channel blocker is amlodipine or a pharmacologically acceptable salt thereof, and

the hydrophilic polymer is at least one compound selected from hydroxypropyl methyl cellulose, methyl cellulose, hydroxypropyl cellulose, sodium carboxymethyl cellulose, macrogol, HA Sankyo, polyvinylpyrrolidone and polyvinyl alcohol,

(28) the pharmaceutical preparation according to (1) or (2) wherein the angiotensin II receptor antagonist is olmesartan medoxomil,

the calcium channel blocker is amlodipine or a pharmacologically acceptable salt thereof, and

the hydrophilic polymer is at least one compound selected from hydroxypropyl methyl cellulose, methyl cellulose, hydroxypropyl cellulose and sodium carboxymethyl cellulose,

(29) the pharmaceutical preparation according to (1) or (2) wherein the angiotensin II receptor antagonist is olmesartan medoxomil,

the calcium channel blocker is amlodipine or a pharmacologically acceptable salt thereof, and

the hydrophilic polymer is either or both of methyl cellulose and hydroxypropyl cellulose,

(30) the pharmaceutical preparation according to (1) or (3) wherein the angiotensin II receptor antagonist is olmesartan medoxomil,

the calcium channel blocker is amlodipine or a pharmacologically acceptable salt thereof, and

the acidic substance is either or both of tartaric acid and ascorbic acid,

(31) the pharmaceutical preparation according to (1) or (4) wherein the angiotensin II receptor antagonist is olmesartan medoxomil,

the calcium channel blocker is amlodipine or a pharmacologically acceptable salt thereof, and

the fluidizing agent is at least one compound selected from calcium silicate, light anhydrous silicic acid, anhydrous calcium hydrogenphosphate, synthetic hydrotalcite and magnesium metasilicate aluminate, and

(32) the pharmaceutical preparation according to (27) to (31) wherein the calcium channel blocker is amlodipine besylate.

According to the present invention, a pharmaceutical preparation, that contains an angiotensin II receptor antagonist and a calcium channel blocker, and which further comprises at least one substance selected from a hydrophilic polymer, an acidic substance and a fluidizing agent, is provided which has improved dissolution properties.

### BEST MODE FOR CARRYING OUT THE INVENTION

The pharmaceutical preparation of the present invention contains an angiotensin II receptor antagonist and a calcium channel blocker as its active ingredients.

Since various medicaments have been proposed as an “angiotensin II receptor antagonist”, which is one of the active ingredients in a solid dosage form of the present invention, and many are actually used clinically, a person of ordinary skill in the art can select suitable medicaments that demonstrate the desired effect as an angiotensin II receptor antagonist for use in the present invention. Suitable, non-limiting examples of angiotensin II receptor antagonists for use in the present invention include losartan (preferably losartan potassium), candesartan cilexetil, valsartan, telmisartan, pratosartan, olmesartan medoxomil and irbesartan. Of these, olmesartan medoxomil is preferably used. Olmesartan medoxomil can easily be produced according to the methods disclosed in the art, suitable examples including the methods disclosed in Japanese Patent No. 2082519 (corresponding to U.S. Pat. No. 5,616,599).

Since various medicaments have been proposed as a “calcium channel blocker”, which is one of the active ingredients in a solid dosage form of the present invention, and many are actually used clinically, a person of ordinary skill in the art can select suitable medicaments that demonstrate the desired effect as a calcium channel blocker for use in the present invention. Suitable, non-limiting examples of calcium channel blockers for use in the present invention include nifedipine, nimodipine, nilvadipine, manidipine (preferably manidipine hydrochloride), barnidipine (preferably barnidipine hydrochloride), nitrendipine, benidipine (preferably benidipine hydrochloride), nicardipine (preferably nicardipine hydrochloride), lercanidipine (preferably lercanidipine hydrochloride), amlodipine (preferably amlodipine besylate), nisoldipine, efonidipine (preferably efonidipine hydrochloride), cilnidipine, azelnidipine, felodipine, aranidipine and pranidipine. Of these amlodipine besylate is preferably used. Amlodipine and its salts including amlodipine besylate can be easily produced according to the methods disclosed in the art, suitable examples including the methods disclosed in Japanese Patent No. 1401088 (corresponding to U.S. Pat. No. 4,572,909).

The pharmacologically acceptable salts of angiotensin II receptor antagonists and a calcium channel blockers described above are not specifically restricted and these salts can be selected by a person of ordinary skill in the art. Suitable pharmacologically acceptable salts include, for example, an alkaline metal salt such as a sodium salt, potassium salt or lithium salt; an alkaline earth metal salt such as a calcium salt or magnesium salt; a metal salt such as an aluminium salt, iron salt, zinc salt, copper salt, nickel salt or cobalt salt; an amine salt such as an ammonium salt, t-octylamine salt, dibenzylamine salt, morpholine salt, glucosamine salt, phenylglycine alkyl ester salt, ethylenediamine salt, N-methylglucamine salt, guanidine salt, diethylamine salt, triethylamine salt, dicyclohexylamine salt, N,N′-dibenzylethylenediamine salt, chloroprocaine salt, procaine salt, diethanolamine salt, N-benzyl-phenethylamine salt, piperazine salt, tetramethylammonium salt or tris(hydroxymethyl)aminomethane salt; a hydrohalogenic acid salt such as a hydrofluoride, hydrochloride, hydrobromide or hydroiodide; a nitrate; a perchlorate; a sulfate; a phosphate; a C₁-C₄ alkanesulfonic acid salt, which may be optionally substituted with a halogen atom(s) such as a methanesulfonate, trifluoromethanesulfonate or ethanesulfonate; a C₆-C₁₀ arylsulfonic acid salt, which may be optionally substituted with a C₁-C₄ alkyl group(s), such as a benzenesulfonate or p-toluenesulfonate; a C₁-C₆ aliphatic acid salt such as an acetate, malate, fumarate, succinate, citrate, tartrate, oxalate or maleate; or an amino acid salt such as a glycine salt, lysine salt, arginine salt, ornithine salt, glutamic acid salt or aspartic acid salt.

The pharmacologically acceptable esters of the angiotensin II receptor antagonists described above are not particularly restricted, and can be selected by a person of ordinary skill in the art. In the case of said esters, it is preferable that such esters can be cleaved by a biological process such as hydrolysis in vivo. The group constituting the esters (the group shown as R when the esters thereof are expressed as —COOR) can be, for example, a C₁-C₄ alkoxy C₁-C₄ alkyl group such as methoxyethyl, 1-ethoxyethyl, 1-methyl-1-methoxyethyl, 1-(isopropoxy)ethyl, 2-methoxyethyl, 2-ethoxyethyl, 1,1-dimethyl-1-methoxymethyl, ethoxymethyl, propoxymethyl, isopropoxymethyl, butoxymethyl or t-butoxymethyl; a C₁-C₄ alkoxylated C₁-C₄ alkoxy C₁-C₄ alkyl group such as 2-methoxyethoxymethyl; a C₆-C₁₀ aryloxy C₁-C₄ alkyl group such as phenoxymethyl; a halogenated C₁-C₄ alkoxy C₁-C₄ alkyl group such as 2,2,2-trichloroethoxymethyl or bis(2-chloroethoxy)methyl; a C₁-C₄ alkoxycarbonyl C₁-C₄ alkyl group such as methoxycarbonylmethyl; a cyano C₁-C₄ alkyl group such as cyanomethyl or 2-cyanoethyl; a C₁-C₄ alkylthiomethyl group such as methylthiomethyl or ethylthiomethyl; a C₆-C₁₀ arylthiomethyl group such as phenylthiomethyl or naphthylthiomethyl; a C₁-C₄ alkylsulfonyl C₁-C₄ lower alkyl group, which may be optionally substituted with a halogen atom(s) such as 2-methanesulfonylethyl or 2-trifluoromethanesulfonylethyl; a C₆-C₁₀ arylsulfonyl C₁-C₄ alkyl group such as 2-benzenesulfonylethyl or 2-toluenesulfonylethyl; a C₁-C₇ aliphatic acyloxy C₁-C₄ alkyl group such as formyloxymethyl, acetoxymethyl, propionyloxymethyl, butyryloxymethyl, pivaloyloxymethyl, valeryloxymethyl, isovaleryloxymethyl, hexanoyloxymethyl, 1-formyloxyethyl, 1-acetoxyethyl, 1-propionyloxyethyl, 1-butyryloxyethyl, 1-pivaloyloxyethyl, 1-valeryloxyethyl, 1-isovaleryloxyethyl, 1-hexanoyloxyethyl, 2-formyloxyethyl, 2-acetoxyethyl, 2-propionyloxyethyl, 2-butyryloxyethyl, 2-pivaloyloxyethyl, 2-valeryloxyethyl, 2-isovaleryloxyethyl, 2-hexanoyloxyethyl, 1-formyloxypropyl, 1-acetoxypropyl, 1-propionyloxypropyl, 1-butyryloxypropyl, 1-pivaloyloxypropyl, 1-valeryloxypropyl, 1-isovaleryloxypropyl, 1-hexanoyloxypropyl, 1-acetoxybutyl, 1-propionyloxybutyl, 1-butyryloxybutyl, 1-pivaloyloxybutyl, 1-acetoxypentyl, 1-propionyloxypentyl, 1-butyryloxypentyl, 1-pivaloyloxypentyl or 1-pivaloyloxyhexyl; a C₅-C₆ cycloalkylcarbonyloxy C₁-C₄ alkyl group such as cyclopentylcarbonyloxymethyl, cyclohexylcarbonyloxymethyl, 1-cyclopentylcarbonyloxyethyl, 1-cyclohexylcarbonyloxyethyl, 1-cyclopentylcarbonyloxypropyl, 1-cyclohexylcarbonyloxypropyl, 1-cyclopentylcarbonyloxybutyl or 1-cyclohexylcarbonyloxybutyl; a C₆-C₁₀ arylcarbonyloxy C₁-C₄ alkyl group such as benzoyloxymethyl; a C₁-C₆ alkoxycarbonyloxy C₁-C₄ alkyl group such as methoxycarbonyloxymethyl, 1-(methoxycarbonyloxy)ethyl, 1-(methoxycarbonyloxy)propyl, 1-(methoxycarbonyloxy)butyl, 1-(methoxycarbonyloxy)pentyl, 1-(methoxycarbonyloxy)hexyl, ethoxycarbonyloxymethyl, 1-(ethoxycarbonyloxy)ethyl, 1-(ethoxycarbonyloxy)propyl, 1-(ethoxycarbonyloxy)butyl, 1-(ethoxycarbonyloxy)pentyl, 1-(ethoxycarbonyloxy)hexyl, propoxycarbonyloxymethyl, 1-(propoxycarbonyloxy)ethyl, 1-(propoxycarbonyloxy)propyl, 1-(propoxycarbonyloxy)butyl, isopropoxycarbonyloxymethyl, 1-(isopropoxycarbonyloxy)ethyl, 1-(isopropoxycarbonyloxy)butyl, butoxycarbonyloxymethyl, 1-(butoxycarbonyloxy)ethyl, 1-(butoxycarbonyloxy)propyl, 1-(butoxycarbonyloxy)butyl, isobutoxycarbonyloxymethyl, 1-(isobutoxycarbonyloxy)ethyl, 1-(isobutoxycarbonyloxy)propyl, 1-(isobutoxycarbonyloxy)butyl, t-butoxycarbonyloxymethyl 1-(t-butoxycarbonyloxy)ethyl, pentyloxycarbonyloxymethyl, 1-(pentyloxycarbonyloxy)ethyl, 1-(pentyloxycarbonyloxy)propyl, hexyloxycarbonyloxymethyl, 1-(hexyloxycarbonyloxy)ethyl or 1-(hexyloxycarbonyloxy)propyl; a C₅-C₆ cycloalkyloxycarbonyloxy C₁-C₄ alkyl group such as cyclopentyloxycarbonyloxymethyl, 1-(cyclopentyloxycarbonyloxy)ethyl, 1-(cyclopentyloxycarbonyloxy)propyl, 1-(cyclopentyloxycarbonyloxy)butyl; cyclohexyloxycarbonyloxymethyl, 1-(cyclohexyloxycarbonyloxy)ethyl, 1-(cyclohexyloxycarbonyloxy)propyl or 1-(cyclohexyloxycarbonyloxy)butyl; a [5-(C₁-C₄ alkyl)-2-oxo-1,3-dioxolen-4-yl]methyl group such as (5-methyl-2-oxo-1,3-dioxolen-4-yl)methyl, (5-ethyl-2-oxo-1,3-dioxolen-4-yl)methyl, (5-propyl-2-oxo-1,3-dioxolen-4-yl)methyl, (5-isopropyl-2-oxo-1,3-dioxolen-4-yl)methyl or (5-butyl-2-oxo-1,3-dioxolen-4-yl), methyl; a [5-(phenyl, which may be optionally substituted with a C₁-C₄ alkyl, C₁-C₄ alkoxy or halogen atom(s))-2-oxo-1,3-dioxolen-4-yl]methyl group such as (5-phenyl-2-oxo-1,3-dioxolen-4-yl)methyl, [5-(4-methylphenyl)-2-oxo-1,3-dioxolen-4-yl]methyl, [5-(4-methoxyphenyl)-2-oxo-1,3-dioxolen-4-yl]methyl, [5-(4-fluorophenyl)-2-oxo-1,3-dioxolen-4-yl]methyl or [5-(4-chlorophenyl)-2-oxo-1,3-dioxolen-4-yl]methyl; or a phthalidyl group, which may be optionally substituted with a C₁-C₄ alkyl or C₁-C₄ alkoxy group(s), such as phthalidyl, dimethylphthalidyl or dimethoxyphthalidyl.

The “hydrophilic polymers” in the pharmaceutical preparations of the present invention are polymers that have an affinity for water. Preferred “hydrophilic polymers” for use in the present invention are ones which are water-soluble. Suitable, non-limiting examples of hydrophilic polymers for use in the present invention include cellulose derivatives such as hydroxypropyl methyl cellulose, methyl cellulose, hydroxypropyl cellulose and sodium carboxymethyl cellulose; synthetic polymers such as polyvinylpyrrolidone, aminoalkyl methacrylate copolymer, carboxyvinyl polymer, polyvinyl alcohol and macrogol (i.e. polyethylene glycol); HA Sankyo (a pre-mixed coating agent comprising a mixture of 16-26% by weight of polyvinyl acetal diethyl aminoacetate, 50-75% by weight of hydroxypropylmethyl cellulose 2910, 12-17% by weight of stearic acid and 1.5-2.3% by weight of fumaric acid), gum Arabic, agar, gelatin and sodium alginate. Of these, hydroxypropyl methyl cellulose, methyl cellulose, hydroxypropyl cellulose, sodium carboxymethyl cellulose, macrogol, HA Sankyo, polyvinylpyrrolidone and polyvinyl alcohol are preferred, hydroxypropyl methyl cellulose, methyl cellulose, hydroxypropyl cellulose, macrogol and sodium carboxymethyl cellulose are more preferred, and methyl cellulose is most preferred. In the present invention, these hydrophilic polymers can be used alone or two or more kinds can be used in combination. Where at least one hydrophilic polymer is present in the pharmaceutical preparation of the present invention, said hydrophilic polymer (or polymers) is preferably present in an amount of from 1 to 90% by weight of the total weight of the pharmaceutical preparation, and more preferably from 5 to 85% by weight. The one or more hydrophilic polymers may be uniformly distributed throughout the entire pharmaceutical preparation, or they may be contained in only a part of said pharmaceutical preparation. If one or more film coating layers are present in the pharmaceutical preparation, the one or more hydrophilic polymers may be contained in said film coating layers.

Suitable, non-limiting examples of “acidic substances” for use in the pharmaceutical preparations of the present invention include inorganic acids such as hydrochloric acid, phosphoric acid, potassium dihydrogen phosphate and sodium dihydrogen phosphate; organic acids such as citric acid, lactic acid, tartaric acid, fumaric acid, phthalic acid, acetic acid, oxalic acid, malonic acid, adipic acid, phytic acid, succinic acid, glutaric acid, maleic acid, malic acid, mandelic acid, ascorbic acid, benzoic acid, methanesulfonic acid, capric acid, caproic acid, caprylic acid, lauric acid, arachic acid, erucic acid, linoleic acid, linolenic acid, oleic acid, palmitic acid, myristic acid and stearic acid; and amino acids such as aspartic acid, L-glutamic acid, L-cysteine, arginine hydrochloride, lysine hydrochloride and L-glutamic acid hydrochloride. Of these, tartaric acid and ascorbic acid are particularly preferred. In the present invention, these acidic substances can be used alone or two or more kinds can be used in combination. Where at least one acidic substance is present in the pharmaceutical preparation of the present invention, said acidic substance (or acidic substances) is preferably present in an amount of from 1 to 90% by weight of the total weight of the pharmaceutical preparation, and more preferably from 5 to 85% by weight.

The “fluidizing agents” in the pharmaceutical preparations of the present invention are flow-modifying agents that increase the fluidity of the other ingredients present in said preparations. Suitable, non-limiting examples of “fluidizing agents” for use in the pharmaceutical preparations of the present invention include hydrous silicon dioxide, light anhydrous silicic acid, microcrystalline cellulose, synthetic aluminum silicate, alumina, magnesium hydroxide, stearic acid, calcium stearate, magnesium stearate, tribasic calcium phosphate, talc, concentrated glycerin, Perfiller 101, anhydrous ethanol, calcium silicate, anhydrous calcium hydrogenphosphate, synthetic hydrotalcite and magnesium metasilicate aluminate. Of these, calcium silicate, light anhydrous silicic acid, anhydrous calcium hydrogenphosphate, synthetic hydrotalcite and magnesium metasilicate aluminate are preferred. In the present invention, these can be used alone or two or more kinds can be used in combination. Where at least one fluidizing agent is present in the pharmaceutical preparation of the present invention, said fluidizing agent (or fluidizing agents) is preferably present in an amount of from 1 to 90% by weight of the total weight of the pharmaceutical preparation, and more preferably from 5 to 85% by weight.

The pharmaceutical preparation of the present invention can where desired additionally contain at least one further additive such as a suitable pharmacologically acceptable excipient, lubricant, binder, disintegrant, emulsifier, stabilizer, corrective or diluent.

Suitable “excipients” include organic excipients including sugar derivatives such as lactose, sucrose, glucose, mannitol or sorbitol; starch derivatives such as corn starch, potato starch, α-starch or dextrin; cellulose derivatives such as microcrystalline cellulose; gum Arabic; dextran; and pullulan, and inorganic excipients including silicate derivatives such as light anhydrous silicic acid, synthetic aluminum silicate, calcium silicate or magnesium metasilicate aluminate; phosphates such as dibasic calcium hydrogenphosphate; carbonates such as calcium carbonate; and sulfates such as calcium sulfate.

Suitable “lubricants” include stearic acid; stearic acid metal salts such as calcium stearate or magnesium stearate; talc; colloidal silica; waxes such as beeswax or spermaceti; boric acid; adipic acid; sulfates such as sodium sulfate; glycol; fumaric acid; sodium benzoate; D,L-leucine; lauryl sulfates such as sodium lauryl sulfate or magnesium lauryl sulfate; silicates such as silicic anhydride or silicate hydrate; and the aforementioned starch derivatives.

Suitable “binders” include hydroxypropyl cellulose, hydroxypropyl methyl cellulose, polyvinylpyrrolidone, macrogol and compounds similar to the aforementioned excipients.

Suitable “disintegrants” include cellulose derivatives such as low-substituted hydroxypropyl cellulose, carboxymethyl cellulose, calcium carboxymethyl cellulose or internally crosslinked sodium carboxymethyl cellulose; cross-linked polyvinylpyrrolidone; and chemically modified starches/celluloses such as carboxymethyl starch or sodium carboxymethyl starch.

Suitable “emulsifiers” include colloidal clays such as bentonite or bee gum; metal hydroxides such as magnesium hydroxide or aluminum hydroxide; anionic surfactants such as sodium lauryl sulfate or calcium stearate; cationic surfactants such as benzalkonium chloride; and nonionic surfactants such as polyoxyethylene alkyl ether, polyoxyethylene sorbitan fatty acid ester or sucrose fatty acid ester.

Suitable “stabilizers” include para-hydroxybenzoic acid esters such as methyl paraben or propyl paraben; alcohols such as chlorobutanol, benzyl alcohol or phenyl ethyl alcohol; benzalkonium chloride; phenols such as phenol or cresol; thimerosal; dehydroacetic acid; and sorbic acid.

Suitable “correctives” include sweeteners such as sodium saccharin or aspartame; sour flavourings such as citric acid, malic acid or tartaric acid; and fragrances such as menthol, lemon or orange fragrance.

Suitable “diluents” include lactose, mannitol, glucose, sucrose, calcium sulfate, calcium phosphate, hydroxypropyl cellulose, microcrystalline cellulose, water, ethanol, polyethylene glycol, propylene glycol, glycerol, starch, polyvinylpyrrolidone, magnesium metasilicate aluminate, and mixtures thereof.

The pharmaceutical preparation of the present invention may be a preparation in which the angiotensin II receptor antagonist and the calcium channel blocker are provided in separate pharmaceutical dosage forms (e.g. solid dosage forms), one or both of which further comprise one or more substances selected from hydrophilic polymers, acidic substances and fluidizing agents, i.e. the pharmaceutical preparation is provided as a “kit of parts” wherein the separate pharmaceutical dosage forms are either administered to the patient together or at a suitable time interval such that they are able to act together in the desired manner. Alternatively, the pharmaceutical preparation of the present invention comprises the angiotensin II receptor antagonist, the calcium channel blocker and the one or more substances selected from hydrophilic polymers, acidic substances and fluidizing agents are provided together in a single dosage form. Preferably, the pharmaceutical preparation of the present invention is a single dosage form.

The pharmaceutical preparation of the present invention is preferably a solid dosage form, either as separate solid dosage forms in a “kit of parts” or, preferably, as a single solid dosage form. Suitable solid dosage forms will be well known to the person skilled in the art, and non-limiting examples of the solid dosage form of the present invention include tablets (including sublingual tablets and tablets that disintegrate in the mouth), capsules (including soft capsules and microcapsules), granules, grains, powders, pills and lozenges. Of these, powders, grains, granules, capsules and tablets are preferred, and tablets are most preferred.

A dosage form of the present invention may be produced using any commonly used method well known to persons skilled in the art of pharmaceutical formulation technology and there are no particular limitations thereon. Examples of suitable methods include those disclosed in publications such as Powder Technology and Pharmaceutical Processes [D. Chulia et al., Elsevier Science Pub. Co. (Dec. 1, 1993)].

A tablet of the present invention can be obtained, for example, by granulating, drying and sizing a principal agent with a vehicle, binder and so forth using a suitable method well known in the art, adding a lubricant and so forth to the resulting mixture followed by mixing and forming into a tablet. Granulation can be carried out by any suitable method well known in the art such as wet granulation, dry granulation or heated granulation. Suitable, non-limiting examples include these granulation techniques carried out using a high-speed agitation granulator, a fluidized granulation dryer, an extrusion granulator or a roller compactor. In addition, procedures such as drying and sizing may be carried out as necessary following granulation. A mixture of the principal agent, vehicle, binder, lubricant and so forth can also be directly formed into tablets. Furthermore, a tablet of the present invention may also be provided with at least one layer of a film coating.

If a film coating is desired, any film coating apparatus of a type well known in the art can be used, and as film coating bases, suitable examples include sugar coating bases, hydrophilic film coating bases, enteric film coating bases and sustained release film coating bases.

Suitable examples of sugar coating bases include saccharose, and these can be used in combination with one or more additives such as talc, precipitated calcium carbonate, calcium phosphate, calcium sulfate, gelatin, gum Arabic, polyvinylpyrrolidone and pullulan.

Suitable examples of hydrophilic film coating bases include cellulose derivatives such as hydroxypropyl cellulose, hydroxypropyl methyl cellulose, hydroxyethyl cellulose, methyl hydroxyethyl cellulose and sodium carboxymethyl cellulose; synthetic polymers such as polyvinyl acetal diethyl aminoacetate, aminoalkyl methacrylate copolymer, polyvinylpyrrolidone and macrogol; and polysaccharides such as pullulan.

Suitable examples of enteric film coating bases include cellulose derivatives such as hydroxypropyl methyl cellulose, phthalate hydroxypropyl methyl cellulose acetate succinate, carboxymethyl ethyl cellulose and cellulose acetate phthalate; acrylic acid derivatives such as methacrylic acid copolymer L, methacrylic acid copolymer LD and methacrylic acid copolymer S; and natural substances such as shellac.

Suitable examples of sustained release film coating bases include cellulose derivatives such as ethyl cellulose; and acrylic acid derivatives such as aminoalkyl methacrylate copolymer RS, ethyl acrylate-methyl methacrylate copolymer emulsion.

A mixture of two or more different coating bases such as those above may also be used in a suitable ratio. In addition, the coating films may also contain suitable pharmacologically acceptable additives such as plasticizers, excipients, lubricants, opacifying agents, colorants or antiseptics as necessary.

The doses and the dosing ratios of the angiotensin II receptor antagonist and calcium channel blocker, which are the active ingredients in the pharmaceutical preparation of the present invention, can be changed depending on various factors such as the activity of each of the active ingredients and the symptoms, age and body weight of the patient. Although the dosage varies depending on symptoms, age and the like, the dose of each class of active ingredient in the case of oral administration is typically from 0.001 mg/kg (preferably 0.01 mg/kg) per day as a lower limit to 10 mg/kg (preferably 1 mg/kg) per day as an upper limit for a human adult, and the dosage can be administered from one to six times per day depending on the symptoms of the patients.

In addition, the dosing ratio of the angiotensin II receptor antagonist and calcium channel blocker, which are the active ingredients in the pharmaceutical preparation of the present invention, can also be changed over a wide range. For example, the dosing ratio by weight of angiotensin II receptor antagonist and calcium channel blocker can typically be within a range of 1:1000 to 1000:1, preferably within a range of 1:100 to 100:1, and more preferably within a range of 1:10 to 10:1.

The pharmaceutical preparation of the present invention is effective for the prophylaxis or treatment of, for example, hypertension or diseases caused by hypertension [more specifically, hypertension, heart disease (angina pectoris, myocardial infarction, arrhythmia, cardiac insufficiency or hypercardia), kidney disease (diabetic nephropathy, glomerular nephritis or nephrosclerosis), or cerebrovascular disease (cerebral infarction or cerebral hemorrhage)] and the like.

### EXAMPLES

The present invention will be described in more detail by way of the following examples, but the scope of the present invention is not limited thereto.

### Example 1

Olmesartan medoxomil, amlodipine besylate, lactose, low substituted hydroxypropyl cellulose, microcrystalline cellulose, methyl cellulose and magnesium stearate were each weighed out in the relative amounts given in column 1 of Table 1 below, and they were then mixed for 2 minutes in an agate mortar. The resulting mixture was formed into tablets using a hydraulic single-action tablet press with a stamp having a 7 mm diameter flat surface at a tablet weight of 140 mg and pressing pressure of 10 kN. The dissolution properties of the resulting tablets were tested according to the procedure shown in Test Example 1 below and the results are shown in column 1 of the following Table 2.

### Example 2

A similar procedure to Example 1 was carried out using the relative amounts given in column 2 of Table 1, the methyl cellulose of Example 1 being replaced with hydroxypropyl cellulose. The dissolution properties of the resulting tablets were tested according to the procedure shown in Test Example 1 below and the results are shown in column 2 of the following Table 2.

### Example 3

A similar procedure to Example 1 was carried out using the relative amounts given in column 3 of Table 1, the methyl cellulose of Example 1 being replaced with hydroxypropyl methyl cellulose. The dissolution properties of the resulting tablets were tested according to the procedure shown in Test Example 1 below and the results are shown in column 3 of the following Table 2.

### Reference Example 1

A similar procedure to Example 1 was carried out using the relative amounts given in column 4 of Table 1, in which the methyl cellulose used in Example 1 is excluded and additional lactose is used in its place. The dissolution properties of the resulting tablets were tested according to the procedure shown in Test Example 1 below and the results are shown in column 4 of the following Table 2.

### Test Example 1

Testing for the rate of dissolution of the tablets prepared in Examples 1 to 3 and Reference Example 1 was carried out in accordance with Method 2 of the Dissolution Test (Paddle Method) described in the 14th Revised Edition of the Japanese Pharmacopoeia at 50 revolutions per minute and using 900 mL of Japanese Pharmacopoeia Solution 2 (JP-2) for the test solution. The test solution was sampled at 30 minutes and 60 minutes after the start of testing followed by measurement of the dissolution rate and dissolved amount of olmesartan medoxomil by absorption spectrometry (dissolution tester: Toyama Sangyo; spectrophotometer: Shimadzu). Testing was carried out on two tablets and their average value is indicated in each case.

TABLE 1

| No. | 1 | 2 | 3 | 4 |
|---|---|---|---|---|
| Example/Reference Example | Example 1 | Example 2 | Example 3 | Reference |
| | | | | Example 1 |
| Olmesartan medoxomil | 10 | 10 | 10 | 10 |
| Amlodipine besylate | 13.86 | 13.86 | 13.86 | 13.86 |
| Lactose | 71.14 | 71.14 | 71.14 | 85.14 |
| Low substituted hydroxypropyl | 20 | 20 | 20 | 20 |
| cellulose | | | | |
| Microcrystalline cellulose | 10 | 10 | 10 | 10 |
| Methyl cellulose | 14 | | | |
| Hydroxypropyl cellulose | | 14 | | |
| Hydroxypropyl methyl cellulose | | | 14 | |
| Magnesium stearate | 1 | 1 | 1 | 1 |
| Total (mg/tablet) | 140 | 140 | 140 | 140 |

TABLE 2

| No. | 1 | 2 | 3 | 4 |
|---|---|---|---|---|
| Example/Reference Example | Example 1 | Example 2 | Example 3 | Reference |
| | | | | Example 1 |
| Dissolution rate after 30 minutes (%) | 68.9 | 66.5 | 61.5 | 50.3 |
| (based on a value of 100% for Reference | (137.0%) | (132.2%) | (122.2%) | (100.0%) |
| Example 1) | | | | |
| Amount dissolved after 30 minutes (μg/mL) | 7.7 | 7.4 | 6.8 | 5.6 |
| Dissolution rate after 60 minutes (%) | 77.5 | 76.0 | 70.2 | 56.5 |
| (based on a value of 100% for Reference | (137.2%) | (134.5%) | (124.2%) | (100.0%) |
| Example 1) | | | | |
| Amount dissolved after 60 minutes (μg/mL) | 8.6 | 8.4 | 7.8 | 6.3 |

### Example 4

A similar procedure to Example 1 was carried out using the relative amounts given in column 1 of Table 3, the methyl cellulose of Example 1 being replaced with tartaric acid. The dissolution properties of the resulting tablets were tested according to the procedure shown in Test Example 2 below and the results are shown in column 1 of the following Table 4.

### Example 5

A similar procedure to Example 1 was carried out using the relative amounts given in column 2 of Table 3, the methyl cellulose of Example 1 being replaced with ascorbic acid. The dissolution properties of the resulting tablets were tested according to the procedure shown in Test Example 2 below and the results are shown in column 2 of the following Table 4.

### Example 6

A similar procedure to Example 1 was carried out using the relative amounts given in column 3 of Table 3, the methyl cellulose of Example 1 being replaced with calcium silicate. The dissolution properties of the resulting tablets were tested according to the procedure shown in Test Example 2 below and the results are shown in column 3 of the following Table 4.

### Example 7

A similar procedure to Example 1 was carried out using the relative amounts given in column 4 of Table 3, the methyl cellulose of Example 1 being replaced with light anhydrous silicic acid. The dissolution properties of the resulting tablets were tested according to the procedure shown in Test Example 2 below and the results are shown in column 4 of the following Table 4.

### Example 8

A similar procedure to Example 1 was carried out using the relative amounts given in column 5 of Table 3, the methyl cellulose of Example 1 being replaced with anhydrous calcium hydrogenphosphate. The dissolution properties of the resulting tablets were tested according to the procedure shown in Test Example 2 below and the results are shown in column 5 of the following Table 4.

### Example 9

A similar procedure to Example 1 was carried out using the relative amounts given in column 6 of Table 3, the methyl cellulose of Example 1 being replaced with synthetic hydrotalcite. The dissolution properties of the resulting tablets were tested according to the procedure shown in Test Example 2 below and the results are shown in column 6 of the following Table 4.

### Example 10

A similar procedure to Example 1 was carried out using the relative amounts given in column 7 of Table 3, the methyl cellulose of Example 1 being replaced with magnesium metasilicate aluminate. The dissolution properties of the resulting tablets were tested according to the procedure shown in Test Example 2 below and the results are shown in column 7 of the following Table 4.

### Reference Example 2

A similar procedure to Example 1 was carried out using the relative amounts given in column 8 of Table 3, in which the methyl cellulose from Example 1 is excluded and additional lactose is used in its place. The dissolution properties of the resulting tablets were tested according to the procedure shown in Test Example 2 below and the results are shown in column 8 of the following Table 4.

### Test Example 2

Testing for the rate of dissolution of the tablets prepared in Examples 4 to 10 and Reference Example 2 was carried out in accordance with Method 2 of the Dissolution Test (Paddle Method) described in the 14th Revised Edition of the Japanese Pharmacopoeia at 50 revolutions per minute and using 900 mL of Japanese Pharmacopoeia Solution 2 (JP-2) for the test solution. The test solution was sampled at 30 minutes and 60 minutes after the start of testing followed by measurement of the dissolution rate and dissolved amount of olmesartan medoxomil by absorption spectrometry (dissolution tester: Toyama Sangyo; spectrophotometer: Shimadzu). Testing was carried out on two tablets and their average value is indicated.

TABLE 3

| No. | 1 | 2 | 3 | 4 | 5 | 6 | 7 | 8 |
|---|---|---|---|---|---|---|---|---|
| Example/Reference Example | Example 4 | Example 5 | Example 6 | Example 7 | Example 8 | Example 9 | Example 10 | Reference |
| | Tartaric | Ascorbic | Calcium | Light | Anhydrous | Synthetic | Magnesium | Example 2 |
| | acid | acid | silicate | anhydrous | dibasic calcium | hydrotalcite | metasilicate | |
| | | | | silicic acid | phosphate | | aluminate | |
| Olmesartan medoxomil | 10 | 10 | 10 | 10 | 10 | 10 | 10 | 10 |
| Amlodipine besylate | 13.86 | 13.86 | 13.86 | 13.86 | 13.86 | 13.86 | 13.86 | 13.86 |
| Lactose | 75.14 | 75.14 | 75.14 | 75.14 | 75.14 | 75.14 | 75.14 | 85.14 |
| Low substituted hydroxypropyl | 20 | 20 | 20 | 20 | 20 | 20 | 20 | 20 |
| cellulose | | | | | | | | |
| Microcrystalline cellulose | 10 | 10 | 10 | 10 | 10 | 10 | 10 | 10 |
| Methyl cellulose | | | | | | | | |
| Hydroxypropyl cellulose | | | | | | | | |
| Hydroxypropyl methyl cellulose | | | | | | | | |
| Various additives | 10 | 10 | 10 | 10 | 10 | 10 | 10 | |
| Magnesium stearate | 1 | 1 | 1 | 1 | 1 | 1 | 1 | 1 |
| Total (mg/tablet) | 140 | 140 | 140 | 140 | 140 | 140 | 140 | 140 |

TABLE 4

| No. | 1 | 2 | 3 | 4 | 5 | 6 | 7 | 8 |
|---|---|---|---|---|---|---|---|---|
| Example/Reference Example | Example 4 | Example 5 | Example 6 | Example 7 | Example 8 | Example 9 | Example 10 | Reference |
| | | | | | | | | Example 2 |
| Dissolution rate after 30 minutes (%) | 89.5 | 83.1 | 92.5 | 88.7 | 81.7 | 59.7 | 92.7 | 50.3 |
| (based a value of 100% for Reference | (177.9%) | (165.2%) | (183.9%) | (176.3%) | (162.4%) | (118.7%) | (184.3%) | (100.0%) |
| Example 1) | | | | | | | | |
| Amount dissolved after 30 minutes | 9.9 | 9.2 | 10.3 | 9.9 | 9.1 | 6.6 | 10.3 | 5.6 |
| (μg/mL) | | | | | | | | |
| Dissolution rate after 60 minutes (%) | 91.7 | 87.5 | 95.2 | 92.1 | 89.2 | 69.9 | 95.6 | 56.5 |
| (based on a value of 100% for | (162.3%) | (154.9%) | (168.5%) | (163.0%) | (157.9%) | (123.7%) | (169.2%) | (100.0%) |
| Reference Example 1) | | | | | | | | |
| Amount dissolved after 60 minutes | 10.2 | 9.7 | 10.6 | 10.2 | 9.9 | 7.8 | 10.6 | 6.3 |
| (μg/mL) | | | | | | | | |

As shown in Tables 2 and 4 above, the pharmaceutical preparations of the present invention demonstrate superior dissolution properties for the angiotensin II receptor antagonist (olmesartan medoxomil) contained therein.

### INDUSTRIAL APPLICABILITY

According to the present invention a pharmaceutical composition, comprising an angiotensin II receptor antagonist and a calcium channel blocker, and further comprising at least one substance selected from hydrophilic polymers, acidic substances and fluidizing agents, is provided which has improved dissolution properties.

What is claimed is:

1. A pharmaceutical preparation comprising a pharmacologically effective amount of a combination of active ingredients comprising an angiotensin II receptor antagonist, a calcium channel blocker and at least one substance selected from the group consisting of a hydrophilic polymer, an acidic substance and a fluidizing agent, said fluidizing agent being a flow-modifying agent that increases the fluidity of said active ingredients.

2. A pharmaceutical preparation according to claim 1, wherein said at least one substance is a hydrophilic polymer.

3. A pharmaceutical preparation according to claim 1, wherein said at least one substance is an acidic substance.

4. A pharmaceutical preparation according to claim 1, wherein said at least one substance is a fluidizing agent.

5. The pharmaceutical preparation according to claim 1, wherein the angiotensin II receptor antagonist is losartan, candesartan, valsartan, telmisartan, pratosartan, olmesartan or irbesartan or a pharmacologically acceptable salt or ester thereof.

6. The pharmaceutical preparation according to claim 1, wherein the angiotensin II receptor antagonist is losartan, candesartan cilexetil, valsartan, telmisartan, pratosartan, olmesartan medoxomil or irbesartan.

7. The pharmaceutical preparation according to claim 1, wherein the angiotensin II receptor antagonist is olmesartan medoxomil.

8. The pharmaceutical preparation according to claim 1, wherein the calcium channel blocker is nifedipine, nimodipine, nilvadipine, manidipine, barnidipine, nitrendipine, benidipine, nicardipine, lercanidipine, amlodipine, nisoldipine, efonidipine, cilnidipine, azelnidipine, felodipine, aranidipine or pranidipine or a pharmacologically acceptable salt thereof.

9. The pharmaceutical preparation according to claim 1, wherein the calcium channel blocker is manidipine, barnidipine, benidipine, nicardipine, lercanidipine, amlodipine, efonidipine or azelnidipine or a pharmacologically acceptable salt thereof.

10. The pharmaceutical preparation according to claim 1, wherein the calcium channel blocker is amlodipine or a pharmacologically acceptable salt thereof.

11. The pharmaceutical preparation according to claim 1, wherein the calcium channel blocker is amlodipine besylate.

12. The pharmaceutical preparation according to claim 2, wherein the hydrophilic polymer is at least one compound selected from the group consisting of a cellulose compound and a synthetic polymer.

13. The pharmaceutical preparation according to claim 2, wherein the hydrophilic polymer is at least one compound selected from the group consisting of a hydroxypropyl methyl cellulose, methyl cellulose, hydroxypropyl cellulose, sodium carboxymethyl cellulose, HA Sankyo, polyvinylpyrrolidone and polyvinyl alcohol.

14. The pharmaceutical preparation according to claim 2, wherein the hydrophilic polymer is at least one cellulose compound.

15. The pharmaceutical preparation according to claim 2, wherein the hydrophilic polymer is at least one compound selected from the group consisting of hydroxypropyl methyl cellulose, methyl cellulose, hydroxypropyl cellulose and sodium carboxymethyl cellulose.

16. The pharmaceutical preparation according to claim 2, wherein the hydrophilic polymer is at least one of methyl cellulose and hydroxypropyl cellulose.

17. The pharmaceutical preparation according to claim 3, wherein the acidic substance is at least one of tartaric acid and ascorbic acid.

18. The pharmaceutical preparation according to claim 4, wherein the fluidizing agent is at least one compound selected from the group consisting of calcium silicate, light anhydrous silicic acid, anhydrous calcium hydrogenphosphate, synthetic hydrotalcite and magnesium metasilicate aluminate.

19. The pharmaceutical preparation according to claim 2, wherein the angiotensin II receptor antagonist is losartan, candesartan cilexetil, valsartan, telmisartan, pratosartan, olmesartan medoxomil or irbesartan,
the calcium channel blocker is amlodipine or a pharmacologically acceptable salt thereof, and
the hydrophilic polymer is at least one compound selected from the group consisting of hydroxypropyl methyl cellulose, methyl cellulose, hydroxypropyl cellulose, sodium carboxymethyl cellulose, HA Sankyo, polyvinylpyrrolidone and polyvinyl alcohol.

20. The pharmaceutical preparation according to claim 2, wherein the angiotensin II receptor antagonist is losartan, candesartan cilexetil, valsartan, telmisartan, pratosartan, olmesartan medoxomil or irbesartan,
the calcium channel blocker is amlodipine besylate, and
the hydrophilic polymer is at least one compound selected from the group consisting of hydroxypropyl methyl cellulose, methyl cellulose, hydroxypropyl cellulose, sodium carboxymethyl cellulose, HA Sankyo, polyvinylpyrrolidone and polyvinyl alcohol.

21. The pharmaceutical preparation according to claim 2, wherein the angiotensin II receptor antagonist is olmesartan medoxomil,
the calcium channel blocker is manidipine, barnidipine, benidipine, nicardipine, lercanidipine, amlodipine, efonidipine or azelnidipine or a pharmacologically acceptable salt thereof, and
the hydrophilic polymer is at least one compound selected from the group consisting of hydroxypropyl methyl cellulose, methyl cellulose, hydroxypropyl cellulose, sodium carboxymethyl cellulose, HA Sankyo, polyvinylpyrrolidone and polyvinyl alcohol.

22. The pharmaceutical preparation according to claim 2, wherein the angiotensin II receptor antagonist is olmesartan medoxomil,
the calcium channel blocker is amlodipine or a pharmacologically acceptable salt thereof, and
the hydrophilic polymer is at least one compound selected from the group consisting of hydroxypropyl methyl cellulose, methyl cellulose, hydroxypropyl cellulose, sodium carboxymethyl cellulose, HA Sankyo, polyvinylpyrrolidone and polyvinyl alcohol.

23. The pharmaceutical preparation according to claim 2, wherein the angiotensin II receptor antagonist is olmesartan medoxomil,
the calcium channel blocker is amlodipine or a pharmacologically acceptable salt thereof, and
the hydrophilic polymer is at least one compound selected from the group consisting of hydroxypropyl methyl cellulose, methyl cellulose, hydroxypropyl cellulose and sodium carboxymethyl cellulose.

24. The pharmaceutical preparation according to claim 2, wherein the angiotensin II receptor antagonist is olmesartan medoxomil,
the calcium channel blocker is amlodipine or a pharmacologically acceptable salt thereof, and
the hydrophilic polymer is at least one of methyl cellulose and hydroxypropyl cellulose.

25. The pharmaceutical preparation according to claim 3, wherein the angiotensin II receptor antagonist is olmesartan medoxomil,
the calcium channel blocker is amlodipine or a pharmacologically acceptable salt thereof, and
the acidic substance is at least one of tartaric acid and ascorbic acid.

26. The pharmaceutical preparation according to claim 4, wherein the angiotensin II receptor antagonist is olmesartan medoxomil,
the calcium channel blocker is amlodipine or a pharmacologically acceptable salt thereof, and
the fluidizing agent is at least one compound selected from the group consisting of calcium silicate, light anhydrous silicic acid, anhydrous calcium hydrogenphosphate, synthetic hydrotalcite and magnesium metasilicate aluminate.

27. The pharmaceutical preparation according to claim 22, wherein the calcium channel blocker is amlodipine besylate.

28. The pharmaceutical preparation according to claim 1, wherein the pharmaceutical preparation is a single dosage form.

29. The pharmaceutical preparation according to claim 28, wherein the single dosage form is a solid dosage form.

30. The pharmaceutical preparation according to claim 29, wherein the solid dosage form is selected from the group consisting of a powder, grains, granules, a capsule and a tablet.

31. The pharmaceutical preparation according to claim 30, wherein the solid dosage form is a tablet.

32. A method for the prophylaxis or treatment of hypertension in a patient in need thereof, said method comprising administering to said patient a pharmacologically effective amount of the pharmaceutical preparation according to claim 1.

33. The method according to claim 32, wherein the patient is a human.

34. The method according to claim 33, wherein the angiotensin II receptor antagonist is olmesartan medoxomil, the calcium channel blocker is amlodipine besylate and the at least one substance is selected from the group consisting of methyl cellulose, hydroxypropyl cellulose, polyvinyl alcohol, tartaric acid, ascorbic acid, calcium silicate, light anhydrous silicic acid, anhydrous calcium hydrogenphosphate, synthetic hydrotalcite and magnesium metasilicate aluminate.

35. A pharmaceutical preparation according to claim 1, wherein the angiotensin II receptor antagonist is olmesartan medoxomil, the calcium channel blocker is amlodipine besylate and the at least one substance is selected from the group consisting of hydroxypropyl methyl cellulose, methyl cellulose, hydroxypropyl cellulose, polyvinyl alcohol, tartaric acid, ascorbic acid, calcium silicate, light anhydrous silicic acid, anhydrous calcium hydrogenphosphate, synthetic hydrotalcite and magnesium metasilicate aluminate.

36. A pharmaceutical preparation according to claim 1, wherein a weight ratio of the angiotensin Ii receptor blocker and the calcium channel blocker is 1:10 to 10:1; and the at least one substance is in an amount of 5 to 85% by weight of the total weight of the pharmaceutical preparation.
